(19) United States
(12) Plant Patent Application Publication
(10) Pub. No.: US 2003/0196237 P1
(43) Pub. Date: Oct. 16, 2003

(54) Chrysanthemum plant named 'Chamon'

(76) Inventor: Jean-Pierre Challet, Nuaille (FR)

Correspondence Address: BURNS, DOANE, SWECKER & MATHIS, L.L.P., P.O. Box 1404, Alexandria, VA, 22313-1404, US

(21) Appl. No.: 10/119,207

(22) Filed: Apr. 10, 2002

(51) Int. Cl. A01H 5/00

(52) U.S. Cl. PLT/291

(57) ABSTRACT

A new and distinct Chrysanthemum cultivar is provided that is the result of a controlled breeding program. Attractive large ball-shaped pink blossoms having pointed ray florets are formed in profusion. Such blossoms commonly are approximately 15 cm in diameter when fully open. The growth habit is stocky and compact and yields a generally spherical overall plant. The foliage is wide and dark green and contrasts well with the pink blossom coloration, and the stems are very strong. The response time is approximately 10 weeks. Exceptional regularity in flowering commonly is displayed. The plant grows well in pots.

### BOTANICAL/COMMERCIAL CLASSIFICATION

Dendranthema grandifloria/decorative Pot Mum

### VARIETAL DENOMINATION

cv. ‘Chamon’

### SUMMARY OF THE INVENTION

The present invention comprises a new and distinct cultivar of Chrysanthemum, botanically known as Dendranthema grandiflora, and hereafter is referred to by the cultivar name ‘Chamon’.

The new cultivar of the present invention was created at Nuaillé, France during the course of a controlled breeding program. The seeds resulting from the cross were sown and small plants were obtained which were physically and biologically different from each other. Selective study resulted in the identification of a single plant of the new variety of the present invention.

It was found that the new Chrysanthemum cultivar of the present invention displays:

(a) a stocky compact growth habit,

(b) forms attractive large long-lasting decorative double ball-shaped pink blossoms having pointed ray florets,

(c) forms very strong stems,

(d) forms attractive wide dark green leaves, and

(e) an ability to grow well in pots.

The new cultivar can be grown singly or in clumps in pots. It also can be grown in the landscape. The pink blossoms contract nicely with the shiny dark green foliage. The blossoms have been found to be long lasting and commonly last approximately four weeks on the plant. Pinching is helpful to further enhance branching.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chamon’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESRIPTION OF THE PHOTOGRAPHS

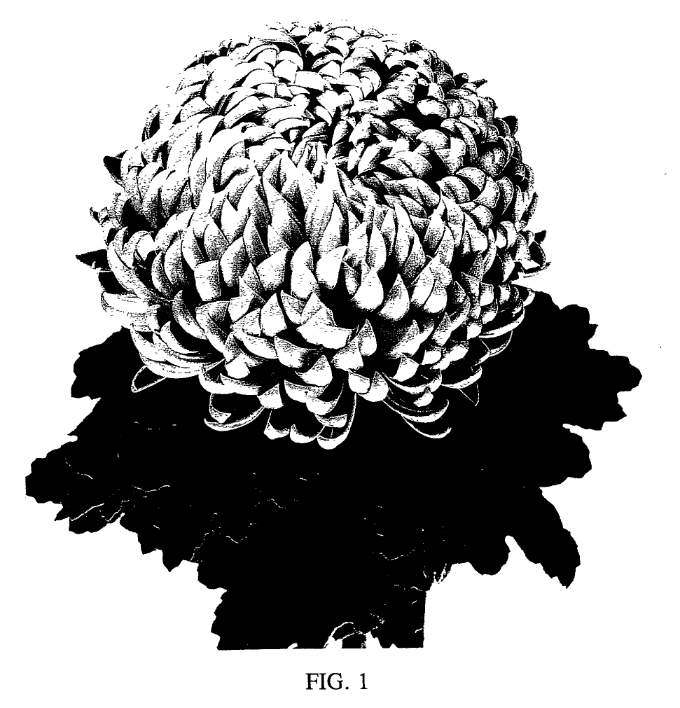

The accompanying FIG. 1 depicts a close view of a typical pink blossom and foliage of the new ‘Chamon’ cultivar while growing in a pot in a greenhouse at Nuaillé, France. The attractive double ball-shaped pink blossom configuration with pointed ray florets is illustrated.

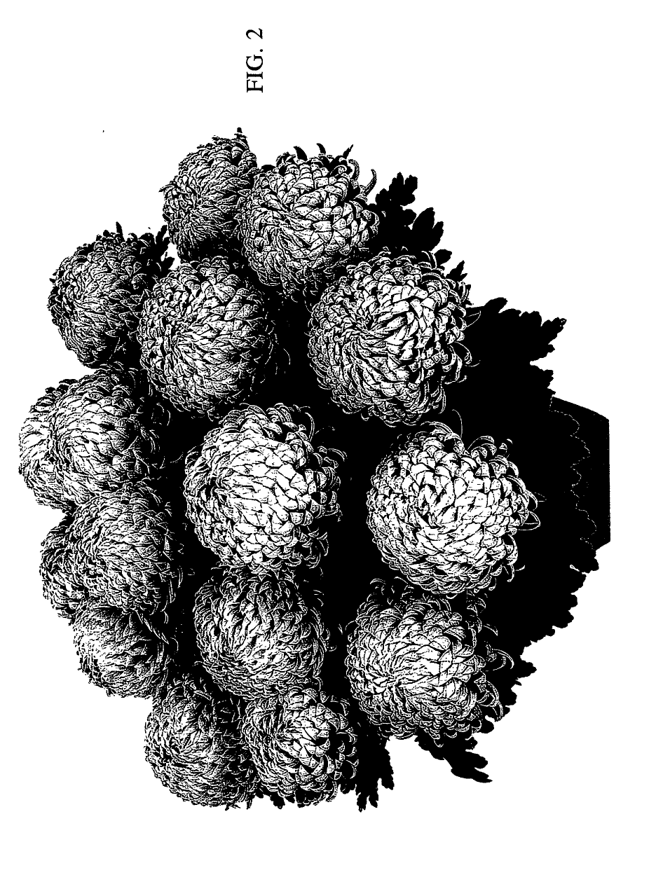

The accompanying FIG. 2 depicts a typical overall plant of the ‘Chamon’ cultivar while growing in a pot in a greenhouse at Nuaillé, France. The stocky compact growth habit is illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General Apperance:

Short and stocky with a generally spherical growth habit.

Foliage:

Attractive dark green and relatively wide.

Stems:

Very strong.

Flowers:

The ray florets are pink in coloration and bear a pointed apex. The disc florets are near green in coloration. The overall configuration is generally ball-shaped and the diameter is large and approximately 15 cm when fully expanded. The flowers are double and bear a large number of ray florets.

Flower Duration:

Very long lasting with the blossoms commonly lasting approximately four weeks on the plant.

Response Time:

The time between the beginning of the short day period and the flowering date at normal temperature and light intensity is approximately ten weeks.

Culture:

Exceptional regularity in flowering.

Usage:

Decorative pot mum.

I claim:

1. A new and distinct cultivar of Chrysanthemum plant that displays:
(a) a stocky compact growth habit,
(b) forms attractive long-lasting decorative ball-shaped pink blossoms having pointed ray florets,
(c) forms very strong stems,
(d) forms attractive wide dark green leaves, and
(e) an ability to grow well in pots;
substantially as illustrated and described.
